(19) United States
(12) Patent Application Publication
(10) Pub. No.: US 2020/0360496 A1
(43) Pub. Date: Nov. 19, 2020

(54) NOVEL COMPOSITION AND USE THEREOF

(71) Applicant: DIAMYD MEDICAL AB, Stockholm (SE)

(72) Inventors: Ulf HANNELIUS, Stockholm (SE); Anders ESSEN-MÖLLER, Stockholm (SE); Anton LINDQVIST, Stockholm (SE)

(21) Appl. No.: 16/964,537

(22) PCT Filed: Feb. 5, 2019
(86) PCT No.: PCT/SE2019/050097

§ 371 Date: Jul. 23, 2020

(30) Foreign Application Priority Data
Feb. 5, 2018 (SE) 1850131-2

(51) Int. Cl. A61K 39/00; A61K 38/51; A61K 38/28; A61K 39/39; A61K 45/06; A61P 37/02

(52) U.S. Cl. CPC A61K 39/0008; A61K 38/51; A61P 37/02; A61K 39/39; A61K 45/06; A61K 38/28

(57) ABSTRACT

The present invention relates to the use of at least two autoantigens specific to an autoimmune disease for treatment of said disease. The invention further relates to a method for treatment or prevention of an autoimmune disease, comprising administering to a subject such a pharmaceutical composition or administering to a subject at least two pharmaceutical compositions, each comprising at least one autoantigen which is not present in another such pharmaceutical composition, and which autoantigen is specific to an autoimmune disease.

### FIELD OF THE INVENTION

The present invention relates to pharmaceutical compositions and their use in therapeutic methods. In particular, the invention relates to composition comprising at least two disease specific autoantigens and the use of such compositions in treatment and/or prevention of autoimmune disease.

### BACKGROUND

Organ specific autoimmune disease is not yet fully understood. Current opinion states that a triggering event, including for example diets, microbiomes, medications, stress, cell dysfunctions, viruses or other pathogens, can set an inflammatory process in motion engaging both the innate and adaptive arms of the immune system in a genetically susceptible individual. Whereas many organ specific autoimmune diseases can be diagnosed prior to onset by identification of for the disease characteristic antibodies, so far a clinical relevant method that effectively can downregulate autoantigen specific inflammation remains to be found.

In autoimmune diabetes (type 1 diabetes and LADA) several beta cell specific autoantigens have been identified including GAD65, pro-insulin, insulin, IA-2 and Znt8. Clinically relevant effects have been obtained in some subgroups of clinical study cohorts using subcutaneous injections of 20 ug GAD65 formulated in alum as adjuvant.

### SUMMARY

According to a first aspect there is provided a pharmaceutical composition comprising at least two autoantigens specific to an autoimmune disease.

According to one embodiment the at least two autoantigens are bound to separate adjuvant particles.

According to another embodiment the at least two autoantigens differ in molecular weight.

According to yet another embodiment, the difference in molecular weight is at least 1:5, such as 1:100, 1:500, or 1:1000.

In yet another embodiment, the separate adjuvant particles for each of the at least two autoantigens are identical or not identical, the latter composition thereby comprising at least two types of adjuvant particles.

In yet another embodiment, the at least two types of adjuvant particles differ in molecular weight and in size.

Furthermore, in one embodiment the adjuvant particles are chosen from the group comprising gold particles, nanoparticles, liposomes, amorphous aluminum hydroxyphosphate sulfate (AAHS), aluminum phosphate, potassium aluminum sulfate and aluminium hydroxide (alum).

In one embodiment the autoimmune disease is selected from the group consisting of type 1 diabetes, Latent Autoimmune Diabetes of Adulthood (LADA), Celiac disease, Multiple Sclerosis, transplant rejection, and Rheumatoid Arthritis.

In one embodiment the at least two autoantigens are selected from the group consisting of GAD, (including GAD65, and GAD67), preproinsulin, proinsulin, an insulin b-chain, Gliadin, hsp65, HSPp277, a MHC molecule from an islet donor cell, islet specific glucose 6 phosphatase catalytic subunit-related protein (IGRP), chromogranin A, insulinoma antigen-2, ZnT8, Myelin Basic Protein (MBP), Myelin Oligodendrocyte Glycoprotein (MOG) or any immunogenic fragments, or nucleic acids encoding such autoantigens.

According to one particular embodiment the pharmaceutical composition comprises GAD and proinsulin.

According to a second aspect, there is provided a method for treatment or prevention of an autoimmune disease, comprising administering to a subject a pharmaceutical composition according to any of the embodiments above; or administering to a subject at least two pharmaceutical compositions, each comprising at least one autoantigen which is not present in another such pharmaceutical composition, and which autoantigen is specific to an autoimmune disease.

According to one embodiment the method for treatment or prevention according to the above comprises administering to a subject at least two pharmaceutical compositions wherein the at least two compositions comprise autoantigens that differ in molecular weight, such as wherein the difference in molecular weight is at least 1:5, such as 1:100, 1:500, or 1:1000.

According to one embodiment of the second aspect, the autoantigens are bound to adjuvant particles, such as gold particles, nano particles, liposomes, amorphous aluminum hydroxyphosphate sulfate (AAHS), aluminum phosphate, potassium aluminum sulfate or aluminium hydroxide (alum).

According to one embodiment of the second aspect, the autoimmune disease is selected from the group consisting of type 1 diabetes, Latent Autoimmune Diabetes of Adulthood (LADA), Celiac disease, Multiple Sclerosis, transplant rejection, and Rheumatoid Arthritis.

According to one embodiment of the second aspect, the at least two autoantigens are selected from the group consisting of GAD, (including GAD65, and GAD67), preproinsulin, proinsulin, an insulin b-chain, Gliadin, hsp65, HSPp277, a MHC molecule from an islet donor cell, isletspecific glucose 6 phosphatase catalytic subunit-related protein (IGRP), chromogranin A, insulinoma antigen-2, ZnT8, Myelin Basic Protein (MBP), Myelin Oligodendrocyte Glycoprotein (MOG) or any immunogenic fragments, or nucleic acids encoding such autoantigens.

According to one embodiment of the second aspect, the autoantigens include GAD and proinsulin.

According to a third aspect there is provided a pharmaceutical composition comprising at least one autoantigen specific for an autoimmune disease, for use in a method for treatment or prevention according to the second aspect and any embodiment thereof.

According to one embodiment, there is provided the pharmaceutical composition according to the above for use in a method for treatment or prevention of an autoimmune disease.

According to one embodiment, the autoimmune disease is an organ-specific autoimmune disease.

According to one embodiment the autoimmune disease is selected from the group consisting of type 1 diabetes, Latent Autoimmune Diabetes of Adulthood (LADA), Celiac disease, Multiple Sclerosis, transplant rejection, and Rheumatoid Arthritis.

According to one embodiment, the method for prevention or treatment of an autoimmune disease comprises administration of the composition or compositions directly into a lymph node, intramuscularly, subcutaneously, intralymphatically, intradermally, intrathetically, intranasally, transbuccally or orally.

According to one embodiment the method for prevention or treatment of an autoimmune disease further comprises administering at least one of Apralozam, AP3 ((Z)-2-Cyano-3-cyclopropyl-N-(4-fluoro-3-methyl-phenyl)-3-hydroxy-thioacrylamide), Lesogaberan, GABA, GABA_{A}-receptor agonists, GABA_{B}-receptor agonists, GABA-PAMs, Vitamin D, anti CD20 antibodies, antiCD3-antibodies, IL-21 antibodies, CTLA4 antibodies, Wharton's Jelly Mesenchymal Stem Cells.

### BRIEF DESCRIPTION OF THE DRAWINGS

FIG. 1 shows the results obtained in the experimental part of the present application.

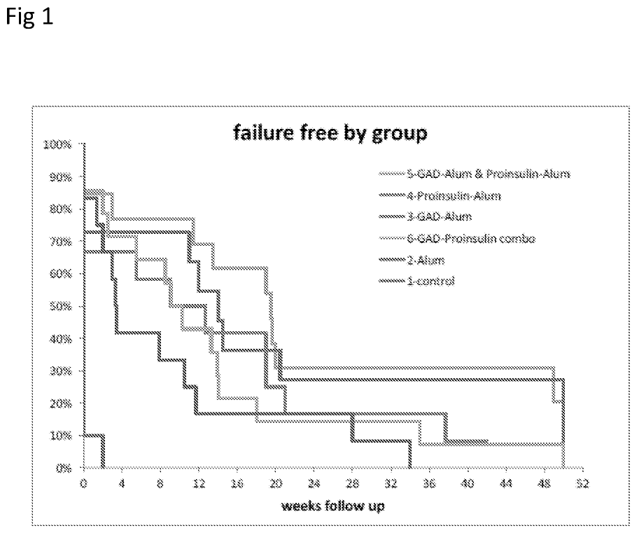

### DEFINITIONS

All terms and expressions as used herein are intended to have the meaning given to them by the person skilled in the art at the filing date of the present application, unless any other expression is evident from the context of this disclosure. However, for the sake of clarity, some terms and expressions are explicitly defined below.

An “autoantigen” is an endogenous tissue constituent that has the ability to interact with autoantibodies and cause an immune response. A “beta cell autoantigen” is an autoantigen originating from pancreatic beta cells.

The term “Vitamin D” includes vitamin D₂ and vitamin D₃. “Vitamin D analogs” include without prejudice Ergocalciferol, Dihydrotachysterol, Alfacalcidol, Calcitriol, Colecalciferol, and Calcifediol, and combinations thereof, as well as any other vitamin D analog classified in group A11CC of the Anatomical Therapeutic Chemical Classification System.

The abbreviation “T1D” stands for “Type 1 Diabetes”.

The abbreviation “GABA” stands for “gamma-amino butyric acid”.

The abbreviation “GAD” stands for “glutamic acid decarboxylase”. GAD is an enzyme that catalyzes the decarboxylation of glutamic acid to GABA and CO₂. GAD exists in two isoforms with molecular weights of 65 (GAD65) and 67 (GAD67) kDa.

The abbreviation “alum” stands for “aluminum hydroxide”.

The abbreviation “PAM” stands for “Positive Allosteric Modulator” and refers to Positive allosteric modulators (PAMs) of GABA_{A} and are well known to those of skill in the art.

Expressions using the singular “a”, “an” and the like shall be construed as including the plural.

### DETAILED DESCRIPTION

Although several compounds have been used to combat autoimmune disease with certain clinical success including not limited to compounds such as ATG, GCSF, anti CD20, antiCD3, antiCD21, antiTNF-alpha anti CTLA-4, cyclosporin, IL2, IL6, such compounds have a general impact on the immune system and do not specifically address autoimmunity and may therefore not be used repeatedly as stand-alone treatment due to the risk of side effects. It seems feasible however to combine reasonably short courses of such compounds with regimens using autoantigens, peptides or nucleic acids coding therefor, for induction of antigen specific tolerance, T-regulatory cells, or for killing or silencing autoantigen specific cytotoxic CD8+ or Central Memory cells.

There is a need in the field to find a way to further improve and extend the clinical results obtained this far.

Recently oral administration of vitamin D (Vit D) combined with three intra-lymphatic doses (4ug) of rhGAD65 adsorbed to alum is shown to preserve endogenous insulin production, reduce requirement of external insulin and lower HbA1c at 30 months (press release by Diamyd Medical AB, 11 Dec. 2017).

Efficacy has been positively correlated with the IL13/IFN-gamma ratio, as well as with the IgG4/IgG1 ratio. Efforts to more directly address the lymph system has also been made without Vit D supplement by means of intradermal administration of proinsulin derived peptide in saline in DR4 restricted new onset type 1 diabetes (T1D) patients and efficacy has been correlated with the IL10/IFN-gamma ratio. In another approach, CD14+Dentritic Cells have been cultivated with vitamin D and Dehexamethazone and subsequently with a proinsulin derived peptide, whereas the educated immature DCs have been administrated by the intradermal route in the lower abdominal quadrants accessing the pancreatic lymph drainage system. Although the intra-lymphatic and intradermal routes are different and may give rise to different immune reactions both routes have been associated with enhanced efficacy as compared with subcutaneous administration.

GABA is a major neurotransmitter and depending on circumstances can exert both stimulatory and inhibitory functions. GABA, GABA_{A}-Receptor agonists, and GABA_{B}-Receptor agonists, and GABA Positive Allosteric Modulators (GABA-PAMs) have been shown to play active roles in downregulating inflammation in autoimmune disease as well as in improving insulin secretion and beta cell replication and stimulating alpha to beta cell transdifferentiation. Recently it has been shown that GABA and autoantigens such as GAD65 and proinsulin-peptide can each be used synergistically with GABA for treatment of autoimmune diabetes in the mouse. It was subsequently hypothesized that using the two autoantigens together in combination with GABA would further enhance the effect on the treatment of autoimmune diabetes.

The present disclosure provides for a pharmaceutical composition comprising at least two autoantigens specific to an autoimmune disease. The at least two autoantigens are preferably bound to separate adjuvant particles. By binding the at least two autoantigens to separate adjuvant particles, the efficacy of the composition is significantly improved, as shown below in the experimental section.

The at least two autoantigens may have the same molecular weight or may differ in molecular weight. Preferably the at least two autoantigens differ in molecular weight. The difference in molecular weight may be at least 1:5, such as 1:100, 1:500, or 1:1000.

The separate adjuvant particles for each of the at least two autoantigens may be identical or not identical. In the case where the separate adjuvant particles for each of the at least two autoantigens are not identical, the composition thereby comprises at least two types of adjuvant particles.

The at least two types of adjuvant particles may differ in molecular weight and in size, or they may have the same or similar molecular weight and size.

The adjuvant particles are chosen from the group comprising gold particles, nanoparticles, liposomes, amorphous aluminum hydroxyphosphate sulfate (AAHS), aluminum phosphate, potassium aluminum sulfate and aluminum hydroxide (alum).

The autoimmune disease is selected from the group consisting of type 1 diabetes, Latent Autoimmune Diabetes of Adulthood (LADA), Celiac disease, Multiple Sclerosis, transplant rejection, and Rheumatoid Arthritis.

The at least two autoantigens are selected from the group consisting of GAD, (including GAD65, and GAD67), preproinsulin, proinsulin, an insulin b-chain, Gliadin, hsp65, HSPp277, a MHC molecule from an islet donor cell, islet specific glucose 6 phosphatase catalytic subunit-related protein (IGRP), chromogranin A, insulinoma antigen-2, ZnT8, Myelin Basic Protein (MBP), Myelin Oligodendrocyte Glycoprotein (MOG) or any immunogenic fragments, or nucleic acids encoding such autoantigens.

Preferably, the pharmaceutical composition comprises GAD and proinsulin.

The present disclosure further provides for a method for treatment or prevention of an autoimmune disease, comprising administering to a subject a pharmaceutical composition according to the above; or administering to a subject at least two pharmaceutical compositions, each comprising at least one autoantigen which is not present in another such pharmaceutical composition, and which autoantigen is specific to an autoimmune disease.

The method for treatment or prevention may comprise administering to a subject at least two pharmaceutical compositions wherein the at least two compositions comprise autoantigens that differ in molecular weight, such as wherein the difference in molecular weight is at least 1:5, such as 1:100, 1:500, or 1:1000.

In the method for treatment or prevention according to the above, the autoantigens are bound to adjuvant particles, such as gold particles, nano particles, liposomes, amorphous aluminum hydroxyphosphate sulfate (AAHS), aluminum phosphate, potassium aluminum sulfate or aluminum hydroxide (alum).

The autoimmune disease may be selected from the group consisting of type 1 diabetes, Latent Autoimmune Diabetes of Adulthood (LADA), Celiac disease, Multiple Sclerosis, transplant rejection, and Rheumatoid Arthritis.

In the method for treatment or prevention according to the above, the at least two autoantigens may be selected from the group consisting of GAD, (including GAD65, and GAD67), preproinsulin, proinsulin, an insulin b-chain, Gliadin, hsp65, HSPp277, a MHC molecule from an islet donor cell, islet specific glucose 6 phosphatase catalytic subunit-related protein (IGRP), chromogranin A, insulinoma antigen-2, ZnT8, Myelin Basic Protein (MBP), Myelin Oligodendrocyte Glycoprotein (MOG) or any immunogenic fragments, or nucleic acids encoding such autoantigens.

In the method for treatment or prevention according to the above, the autoantigens may preferably include GAD and proinsulin.

The present disclosure further provides for a pharmaceutical composition comprising at least one, such as a single, autoantigen specific for an autoimmune disease, for use in a method for treatment or prevention according to the above. The pharmaceutical composition according to the above may be for use in a method for treatment or prevention of an autoimmune disease.

The autoimmune disease may be an organ-specific autoimmune disease. The autoimmune disease may be selected from the group consisting of type 1 diabetes, Latent Autoimmune Diabetes of Adulthood (LADA), Celiac disease, Multiple Sclerosis, transplant rejection, and Rheumatoid Arthritis.

The pharmaceutical composition for use according to above may be used in a method for prevention or treatment of an autoimmune disease comprising administration of the composition or compositions directly into a lymph node, intramuscularly, subcutaneously, intralymphatically, intradermally, intrathetically, intranasally, transbuccally or orally.

The pharmaceutical composition for use according to above may be used in a method for prevention or treatment of an autoimmune disease further comprising administering at least one of Apralozam, AP3 ((Z)-2-Cyano-3-cyclopropyl-N-(4-fluoro-3-methyl-phenyl)-3-hydroxy-thioacrylamide), Lesogaberan, GABA, GABAA-receptor agonists, GABAB-receptor agonists, GABA-PAMs, Vitamin D, anti CD20 antibodies, antiCD3antibodies, IL-21 antibodies, CTLA4 antibodies, Wharton's Jelly Mesenchymal Stem Cells.

In one embodiment, the methods and pharmaceutical compositions disclosed herein are useful in prevention of transplant rejection, in particular prevention of rejection of newly transplanted beta cells or islets of Langerhans. In one embodiment, the autoantigens GAD and proinsulin are included in the methods and/or compositions for prevention of rejection of newly transplanted beta cells or islets of Langerhans.

In a further aspect, the invention relates to a kit of parts comprising at least two pharmaceutical compositions, each comprising one autoantigen specific to an autoimmune disease.

In one embodiment, the at least two compositions comprise autoantigens that differ in molecular weight, such as wherein the difference in molecular weight is at least 1:5, such as 1:100, 1:500, or 1:1000.

In one embodiment, the autoantigens are bound to adjuvant particles, such as from the group comprising gold particles, liposomes, amorphous aluminum hydroxyphosphate sulfate (AAHS), aluminum phosphate, potassium aluminum sulfate or aluminium hydroxide (alum).

In one embodiment, wherein the autoimmune disease is selected from the group consisting of type 1 diabetes, Latent Autoimmune Diabetes of Adulthood (LADA), Celiac disease, Multiple Sclerosis, transplant rejection, and Rheumatoid Arthritis.

In one embodiment, the at least two autoantigens are selected from the group consisting of GAD, (including GAD65, and GAD67), preproinsulin, proinsulin, insulin, an insulin b-chain, hsp65, HSPp277, a MHC molecule from an islet donor cell, islet specific glucose 6 phosphatase catalytic subunit-related protein (IGRP), chromogranin A, insulinoma antigen-2, ZnT8, and any immunogenic fragment, or nucleic acids encoding such autoantigens.

In a presently preferred embodiment, the at least two autoantigens comprises GAD and proinsulin.

The invention further relates to the above described kit of parts, and its individual parts, for use in the methods for treatment disclosed herein.

The results disclosed in the experimental section shows a significant positive effect being achieved by adsorbing each type of antigen to separate particles. It is beneficial to adsorb different antigens or types of antigens on different particles or types of particles. For instance, an antigen A could be adsorbed to a particle X, and an antigen B could be adsorbed to a particle Y. An advantage of this approach is that the particles may then be of different sizes and adapted to the antigen in question. As the size of the antigens differ, it may be advantageous if also the size of the adjuvant particles to which they are adsorbed differ. Consequently, a smaller antigen may for instance be adsorbed to a smaller particle, whereas a larger antigen may be adsorbed to a larger antigen. This may facilitate the availability of the antigens for activity. It is also possible that the reverse may be efficient, that is that a smaller antigen is adsorbed to a larger particle and a larger antigen adsorbed to a smaller particle. This approach could possibly have an impact on the ratio of antigens administered, in that a larger number of small antigens may be adsorbed to the larger particle, and a smaller number of larger antigens may be adsorbed to the smaller particles.

The adjuvant particles that may be used within this embodiment are chosen from the group comprising gold particles, nano-particles, liposomes, MF59, CpG 1018, AS04, AS01_{B}, amorphous aluminum hydroxyphosphate sulfate (AAHS), aluminum hydroxide (Alum), aluminum phosphate, and potassium aluminum sulfate.

Gold as adjuvant particles are normally gold nanoparticles, thus the size of gold particles as adjuvants normally range from 2-50 nm in diameter. For liposomes as adjuvant particles, the size may range from 40-2500 nm, depending on the type of liposome. Oil-in-water formulations normally range from 50-600 nm in size. M59 is an oil-in-water emulsion adjuvant, and has a particle size of around 160 nm. CpG 1018 is short (22-mer) oligonucleotide sequence containing CpG motifs, with a molecular mass of approximately 7150 Da. AS04 is an adjuvant combination consisting of monophosphoryl lipid A (MPL) and alum at a ratio of 100:1. AS01 is a liposome-based adjuvant that contains 3-O-desacyl-monophosphoryl lipid A, and QS-21 which is a saponin molecule. Aluminium hydroxide adjuvant is comprised of particles with a dimension of 100 nm, while aluminium phosphate particles are around 50 nm. In an aqueous solution, particles of both aluminium salts aggregate to form 1 to 20 μm sized particulates. Aluminium hydroxide and aluminium phosphate can be produced in nanoscale size 200 nm. Only amorphous aluminium hydroxyphosphate sulfate is produced in nanoscale for use in vaccine preparations.

The particle size may be linked to the adsorption efficiency of antigens. Nanoscale aluminium particles can adsorb more antigens compared to traditional aluminium-based adjuvants because of the higher surface-area-to-volume ratio. Moreover, the efficacy of particle uptake by the specialised antigen presenting dendritic cells may be in relation to the particle size, where smaller particles may be more advantageous than larger particles. Thus it is possible to adjust the ratio of the at least two autoantigens by choosing particles with different or same sizes for each of the autoantigens, respectively.

The invention is further explained by reference to the below example which shall not be construed as limiting the scope of the invention, which is that of the appended claims.

### Experiment

The aim of this experiment was to evaluate whether combining GAD immunization with another autoantigen (proinsulin) can enhance the therapy's ability to preserve beta cell function in newly diabetic NOD mice.

About 80% of female NOD mice spontaneously develop diabetes between about 15-28 weeks of age. They provide a model with which to test the ability of immune modulators to preserve beta cell function and/or restore normoglycemia after diabetes onset.

Experimental control groups:
1) Untreated female NOD mice from Taconic
2) 6 mg/g GABA+Alum
3) 6 mg/g GABA+100 ug GAD/Alum
4) 6 mg/g GABA+100 ug Proinsulin/Alum
5) 6 mg/g GABA+100 ug GAD/Alum+100 ug Proinsulin/Alum (separate formulations)
6) 6 mg/g GABA+100 ug GAD/Alum+100 ug Proinsulin/Alum (antigens bound to Alum)

Mice were monitored twice a week for the development of hyperglycemia and more frequently once they showed signs of developing hyperglycemia. When their blood glucose was between 250-300 on two consecutive days, they were randomly assigned to one of the above groups. For immunizations, mice received 100 micrograms of antigen(s) bound to alum at disease onset, and one boost 10 days later. One experimental group received GAD and Proinsulin separately bound to alum, and another group received both antigens bound together on alum. Mice were injected the same day that a consecutive daily blood glucose was >250 mgs/dl. They were also immediately started on GABA (6 ml/ml) through the drinking water. The mice received GABA through their water throughout the study. Water was changed twice a week.

After treatment, the mice were monitored 3 times a week for the recurrence of hyperglycemia. Two consecutive blood glucose readings >300 mgs/dL was considered disease recurrence. Mice with two consecutive blood glucose levels >500 mg/dL were euthanized. The mice were followed for up to 50 weeks posttreatment after which remaining mice were sacrificed and their pancreases harvested and fixed, and their plasma saved away at −80 C for further analysis.

The normally distributed data was tested by ANOVA and Students T test. The categorical data was tested by X2 and Fisher's exact test

### Results

Biostatistical analysis at >50 weeks post-treatment.

Of note, is the median number of weeks that disease was reversed for each group (in yellow in the 2nd chart):

| | |
|---|---|
| Control untreated: | 0.0 |
| Alum + GABA | 3.35 |
| GAD + GABA | 10.85 |
| Proinsulin + GABA | 14.00 |
| Proinsulin + GAD (separate) + GABA | 19.5 |
| Proinsulin + GABA (combined) + GABA | 9.7 |

All the treatment groups displayed significant length of disease reversal relative to the control alum+GABA (6 mgs/ml). See the red P values <0.05. Of the antigen treatments, only the proinsulin+GAD (combined)+GABA was significantly better than the alum+GABA (p=0.022). The proinsulin+GABA came close with a p value of 0.069. GAD/Alum+GABA had a p value of 0.24 vs. alum+GABA. Note that this study used GABA at 6 mgs/ml, rather than at 20 mgs/ml as was published in 2014 (in combination with proinsulin/alum). Using 20 mg/ml in this study could have made protection too good prolonging the time until a difference between treatment arms could be seen.

logrank test p values

| | | | p |
|---|---|---|---|
| group | vs group | stat | value |
| D1-cntl | D2-Alum | 13.27 | 0.0003 |
| D1-cntl | D3-GAD Alum | 10.70 | 0.0011 |
| D1-cntl | D4-Proinsulin Alum | 12.05 | 0.0005 |
| D1-cntl | D5-GAD-Alum | 17.00 | 0.0000 |
| | &Proinsulin-Alum | | |
| D1-cntl | D6-GAD Proinsulin Combo | 16.87 | 0.0000 |
| D2-Alum | D3-GAD Alum | 1.36 | 0.2427 |
| D2-Alum | D4-Proinsulin Alum | 3.29 | 0.0696 |
| D2-Alum | D5-GADAlum&ProAlum | 5.19 | 0.0228 |
| D2-Alum | D6\|-GAD Proinsulin Combo | 1.29 | 0.2552 |
| D3-GAD Alum | D4-Proinsulin Alum | 0.54 | 0.4645 |
| D3-GAD Alum | D5-GAD-Alum | 1.79 | 0.1814 |
| | &Proinsulin-Alum | | |
| D3-GAD Alum | D6-GAD Proinsulin Combo | 0.23 | 0.6351 |
| D4-Proinsulin Alum | D5-GAD-Alum | 0.06 | 0.8093 |
| | &Proinsulin-Alum | | |
| D4-Proinsulin Alum | D6-GAD Proinsulin Combo | 1.48 | 0.2235 |
| D5-GAD-Alum | D6-GAD Proinsulin Combo | 3.03 | 0.0820 |
| &Proinsulin-Alum | | | |

Unexpectedly the study showed that adsorbing the two autoantigens to the same alum particles did not show an enhanced beta cell preservation effect, whereas adsorbing each type of antigen to separate alum particles resulted in a significant positive effect. Without being bound by theory, it is hypothesized that the larger size of the GAD molecule causes DC activities that overshadow those of the smaller proinsulin molecule and that therefore the effect of the smaller molecule is suppressed when the antigens are present on the same alum particles. Antigens of different sizes or charge could therefore preferably be each adsorbed to different alum particles

1. A pharmaceutical composition comprising at least two autoantigens specific to an autoimmune disease.

2. The pharmaceutical composition according to claim 1, wherein the at least two autoantigens are bound to separate adjuvant particles.

3. The pharmaceutical composition according to claim 1, wherein the at least two autoantigens differ in molecular weight.

4. The pharmaceutical composition according to claim 3, wherein the difference in molecular weight is at least 1:5, such as 1:100, 1:500, or 1:1000.

5. The pharmaceutical composition according to claim 2, wherein the separate adjuvant particles for each of the at least two autoantigens are identical or not identical, the latter composition thereby comprising at least two types of adjuvant particles.

6. The pharmaceutical composition according to claim 5, wherein the at least two types of adjuvant particles differ in molecular weight and in size.

7. The pharmaceutical composition according to claim 1 wherein the adjuvant particles are chosen from the group comprising gold particles, nanoparticles, liposomes, amorphous aluminum hydroxyphosphate sulfate (AAHS), aluminum phosphate, potassium aluminum sulfate and aluminium hydroxide (alum).

8. The pharmaceutical composition according to claim 1, wherein the autoimmune disease is selected from the group consisting of type 1 diabetes, Latent Autoimmune Diabetes of Adulthood (LADA), Celiac disease, Multiple Sclerosis, transplant rejection, and Rheumatoid Arthritis.

9. The pharmaceutical composition according to claim 1, wherein the at least two autoantigens are selected from the group consisting of GAD, (including GAD65, and GAD67), preproinsulin, proinsulin, an insulin b-chain, Gliadin, hsp65, HSPp277, a MHC molecule from an islet donor cell, islet specific glucose 6 phosphatase catalytic subunit-related protein (IGRP), chromogranin A, insulinoma antigen-2, ZnT8, Myelin Basic Protein (MBP), Myelin Oligodendrocyte Glycoprotein (MOG) or any immunogenic fragments, or nucleic acids encoding such autoantigens.

10. The pharmaceutical composition according to claim 9, comprising GAD and proinsulin.

11. A method for treatment or prevention of an autoimmune disease, comprising administering to a subject a pharmaceutical composition according to claim 1; or administering to a subject at least two pharmaceutical compositions, each comprising at least one autoantigen which is not present in another such pharmaceutical composition, and which autoantigen is specific to an autoimmune disease.

12. The method for treatment or prevention according to claim 11, comprising administering to a subject at least two pharmaceutical compositions wherein the at least two compositions comprise autoantigens that differ in molecular weight, such as wherein the difference in molecular weight is at least 1:5, such as 1:100, 1:500, or 1:1000.

13. The method for treatment or prevention according to claim 11, wherein the autoantigens are bound to adjuvant particles, such as gold particles, nano particles, liposomes, amorphous aluminum hydroxyphosphate sulfate (AAHS), aluminum phosphate, potassium aluminum sulfate or aluminium hydroxide (alum).

14. The method for treatment or prevention according to claim 11, wherein the autoimmune disease is selected from the group consisting of type 1 diabetes, Latent Autoimmune Diabetes of Adulthood (LADA), Celiac disease, Multiple Sclerosis, transplant rejection, and Rheumatoid Arthritis.

15. The method for treatment or prevention according to claim 11, wherein the at least two autoantigens are selected from the group consisting of GAD, (including GAD65, and GAD67), preproinsulin, proinsulin, an insulin b-chain, Gliadin, hsp65, HSPp277, a MHC molecule from an islet donor cell, isletspecific glucose 6 phosphatase catalytic subunit-related protein (IGRP), chromogranin A, insulinoma antigen-2, ZnT8, Myelin Basic Protein (MBP), Myelin Oligodendrocyte Glycoprotein (MOG) or any immunogenic fragments, or nucleic acids encoding such autoantigens.

16. The method for treatment or prevention according to claim 15, wherein the autoantigens include GAD and proinsulin.

17. A pharmaceutical composition comprising at least one autoantigen specific for an autoimmune disease, for use in a method for treatment or prevention according to claim 11.

18. The pharmaceutical composition according to claim 10 for use in a method for treatment or prevention of an autoimmune disease.

19. The pharmaceutical composition for use according to claim 17, wherein the autoimmune disease is an organ-specific autoimmune disease.

20. The pharmaceutical composition for use according to claim 18, wherein the autoimmune disease is selected from the group consisting of type 1 diabetes, Latent Autoimmune Diabetes of Adulthood (LADA), Celiac disease, Multiple Sclerosis, transplant rejection, and Rheumatoid Arthritis.

21. The pharmaceutical composition for use according to claim 17, wherein the method for prevention or treatment of an autoimmune disease comprises administration of the composition or compositions directly into a lymph node, intramuscularly, subcutaneously, intralymphatically, intradermally, intrathetically, intranasally, transbuccally or orally.

22. The pharmaceutical composition for use according to claim 17, wherein the method for prevention or treatment of an autoimmune disease further comprises administering at least one of Apralozam, AP3 ((Z)-2-Cyano-3-cyclopropyl-N-(4-fluoro-3-methyl-phenyl)-3-hydroxy-thioacrylamide), Lesogaberan, GABA, GABAA-receptor agonists, GABAB-receptor agonists, GABA-PAMs, Vitamin D, anti CD20 antibodies, antiCD3antibodies, IL-21 antibodies, CTLA4 antibodies, Wharton's Jelly Mesenchymal Stem Cells.
